(19) United States
(12) Patent Application Publication
(10) Pub. No.: US 2023/0256156 A1
(43) Pub. Date: Aug. 17, 2023

(54) DECOMPRESSION WRAP

(71) Applicant: KCI Manufacturing Unlimited Company, Westmeath (IE)

(72) Inventors: Jonathan G. REHBEIN, San Antonio, TX (US); Larry Tab RANDOLPH, San Antonio, TX (US); Luke PERKINS, San Antonio, TX (US); Richard M. KAZALA, San Antonio, TX (US)

(73) Assignee: KCI Manufacturing Unlimited Company, Westmeath (IE)

(21) Appl. No.: 18/020,706

(22) PCT Filed: Jul. 26, 2021
(86) PCT No.: PCT/IB2021/056743

§ 371 Date: Feb. 10, 2023

Related U.S. Application Data
(60) Provisional application No. 63/065,724, filed on Aug. 14, 2020.

(51) Int. Cl. A61M 1/00; A61F 13/02

(52) U.S. Cl. CPC A61M 1/915; A61M 1/917; A61M 1/962; A61F 13/0216; A61F 13/0273; A61M 2210/086

(57) ABSTRACT

A decompression wrap includes an occlusive layer formed as a first strip having a longitudinal axis, a manifold layer coupled to the occlusive layer and formed as a second strip extending parallel to the longitudinal axis. The occlusive layer extends beyond the manifold layer in a first lateral direction perpendicular to the longitudinal axis. The decompression wrap also includes an adhesive provided on the occlusive layer and configured to seal a first longitudinal section of the occlusive layer to a second longitudinal section of the occlusive layer when the decompression wrap is wrapped around a tissue site.

### CROSS-REFERENCE TO RELATED APPLICATIONS

This application claims the benefit of priority to U.S. Provisional Application No. 63/065,724, filed on Aug. 14, 2020, which is incorporated herein by reference in its entirety.

### BACKGROUND

Swelling associated with trauma or certain pathologies may cause various medical complications. For example, swelling can cause discomfort and pain, may limit range of motion, or otherwise negatively impact patient quality of life. Swelling may also limit the ability of a medical provider to medically image, view, or access underlying tissue, or may otherwise interfere in the treatment of a patient, and thus may pose an impediment to the healing and recovery of the patient. Systems and devices that facilitate swelling reduction at a tissue site may therefore be advantageous. Tissue sites may also include wounds, for example caused by trauma or surgical incision, which may also benefit from treatment therapies.

The present disclosure relates to therapy systems for providing decompression therapy and/or wound therapy to tissue sites.

### SUMMARY

One implementation of the present disclosure is a decompression wrap. The decompression wrap includes an occlusive layer formed as a first strip having a longitudinal axis, a manifold layer coupled to the occlusive layer and formed as a second strip extending parallel to the longitudinal axis. The occlusive layer extends beyond the manifold layer in a first lateral direction perpendicular to the longitudinal axis without extending beyond the manifold layer in a second lateral direction opposite the first lateral direction. The decompression wrap also includes an adhesive provided on the occlusive layer and configured to seal a first longitudinal section of the occlusive layer to a second longitudinal section of the occlusive layer when the decompression wrap is wrapped around a tissue site.

In some embodiments, the occlusive layer extends beyond the manifold layer in the first lateral direction by less than ten centimeters. The occlusive layer may extend beyond the manifold layer in the first lateral direction by between two centimeters and five centimeters.

In some embodiments, the adhesive is configured to provide a substantially air-tight seal between the first longitudinal section of the occlusive layer and the second longitudinal section of the occlusive layer. In some embodiments, the manifold layer includes an open-celled foam and the occlusive layer is substantially air-impermeable. In some embodiments, manifold layer extends beyond the occlusive layer in a second lateral direction opposite the first lateral direction.

The manifold layer includes a first portion longitudinally offset from a second portion of the manifold layer. In some embodiments, the first portion contacts the second portion when the first longitudinal section of the occlusive layer is sealed to the second longitudinal section of the occlusive layer by the adhesive.

In some embodiments, the decompression wrap includes a patient interface layer coupled to the manifold layer such that the manifold layer is positioned between the patient interface layer and the occlusive layer.

In some embodiments, the decompression wrap includes a double-sided adhesive layer positioned between the manifold layer and the occlusive layer. The double-sided adhesive layer is configured to couple the manifold layer to the occlusive layer.

In some embodiments, the manifold layer is configured to collapse toward the occlusive layer when a negative pressure is established at the manifold layer.

Another implementation of the present disclosure is a negative pressure therapy system. The negative pressure therapy system includes a wrap. The wrap includes an occlusive layer formed as a first strip having a longitudinal axis and a manifold layer coupled to the occlusive layer and formed as a second strip extending parallel to the longitudinal axis. A portion of the occlusive layer extends beyond the manifold layer in a first lateral direction perpendicular to the longitudinal axis, and has a width less than ten centimeters. An adhesive is provided along the portion of the occlusive layer extending beyond the manifold layer. The negative pressure therapy system also includes a pump configured to be placed in fluid communication with the manifold layer and operable to remove air from the manifold layer.

In some embodiments, the width of the portion of the occlusive layer extending beyond the manifold layer is in a range between two centimeters and five centimeters. In some embodiments, the occlusive layer does not extend beyond the manifold layer in a second lateral direction opposite the first lateral direction.

In some embodiments, the wrap is configured to be wrapped around a tissue site such that the a first longitudinal section of the occlusive layer is sealed to a second longitudinal section of the occlusive layer by the adhesive and the manifold layer is positioned between the occlusive layer and the tissue site. The manifold layer includes a first portion longitudinally offset from a second portion of the manifold layer. The first portion may contact the second portion when the first longitudinal section of the occlusive layer is sealed to the second longitudinal section of the occlusive layer by the adhesive.

In some embodiments, the negative pressure therapy system also includes a sealing ring configured to provide a substantially air-tight seal between a first end of the decompression wrap and the tissue site.

In some embodiments, the wrap is configured to be applied to a tissue site such that a chamber is defined between the occlusive layer and the tissue site. The manifold layer may be positioned in the chamber and the pump may be in fluid communication with the chamber. The pump is configured to establish a negative pressure in the chamber by removing air from the chamber. In some embodiments, the manifold layer is configured to collapse away from the tissue site when the negative pressure is established in the substantially airtight volume.

In some embodiments, the manifold layer includes an open-celled foam and the occlusive layer is substantially air-impermeable. In some embodiments, the negative pressure therapy system also includes tubing configured to extend from the manifold layer to the pump such that the pump is in fluid communication with the manifold layer via the pump.

Another implementation of the present disclosure is a method for applying decompression therapy. The method includes obtaining a wrap that includes an occlusive layer and a manifold layer, and spirally wrapping the wrap around and along a tissue site such that the occlusive layer defines a chamber between the occlusive layer and the tissue site. The manifold layer is positioned in the chamber and the chamber has a width greater than a width of the occlusive layer. The method also includes removing air from the chamber with a pump to establish a negative pressure at the manifold layer.

In some embodiments, wrapping the wrap around the tissue site comprises adhering a first section of the occlusive layer to a second section of the occlusive layer. In some embodiments, wrapping the wrap around the tissue site comprises placing a first portion of the manifold layer in contact with a second portion of the manifold layer.

Another implementation of the present disclosure is a kit. The kit includes a plurality of decompression wraps. Each decompression wrap includes an occlusive layer, and a manifold layer coupled to the occlusive layer and extending along the manifold layer. The occlusive layer is coextensive with the manifold layer along a first lateral edge parallel to a longitudinal axis of the occlusive layer and wherein at least a portion of the occlusive layer extends beyond the manifold layer at a second lateral side opposite the first lateral side. The wrap also includes an adhesive provided along the portion of the occlusive layer extending beyond the manifold layer. The adhesive is configured to seal a first portion of the occlusive layer to a second portion of the occlusive layer when the decompression wrap is applied to a tissue site. The kit also includes a pump configured to be placed in fluid communication with the manifold layer and operable to remove air from the manifold layer.

In some embodiments, the plurality of decompression wraps are provided as a continuous roll of material configured to be selectively divided by a user into the plurality of decompression wraps. In some embodiments, each of the plurality of decompression wraps has a predetermined length. In some embodiments, the plurality of decompression wraps have a plurality of different predetermined lengths.

In some embodiments, the kit also includes tubing configured to be coupled to the pump and the wrap by a user. In some embodiments, the tubing is coupled to a connection pad configured to facilitate coupling of the tubing to the wrap by the user. In some embodiments, the kit includes a plurality of units of tubing and a plurality of connection pads.

In some embodiments, the kit includes a sealing cuff configured to facilitate selectively establishing an airtight chamber between one of the plurality of wraps and a patient site. In some embodiments, the kit includes an adhesive sealing strip configured to facilitate selectively establishing an airtight chamber between one of the plurality of wraps and a patient site. The portion of the occlusive layer extending beyond the manifold layer may have a width in a range between two centimeters and five centimeters.

### BRIEF DESCRIPTION OF THE DRAWINGS

FIG. 1 is a perspective view of a negative pressure therapy system including a decompression wrap applied to a tissue site, according to an exemplary embodiment.

FIG. 2 is a close-up perspective view of the decompression wrap of FIG. 1 before application to the tissue site, according to an exemplary embodiment.

FIG. 3 is an exploded view of the decompression wrap of FIGS. 1-2, according to an exemplary embodiment.

FIG. 4 is a storyboard-style illustration of a process for applying the decompression wrap to a tissue site, according to an exemplary embodiment.

### DETAILED DESCRIPTION

Referring generally to the FIGURES, a therapy system for applying a negative pressure (relative to atmospheric pressure) to intact skin extending over, or surrounding, different types of treatment tissue sites (such as, e.g., bone tissue, adipose tissue, muscle tissue, neural tissue, dermal tissue, vascular tissue, connective tissue, cartilage, tendons, ligaments, etc.) is described according to various embodiments. In some embodiments, the therapy system disclosed herein is also suitable for applying a negative pressure over wounds (e.g., non-intact skin, surgical incisions, traumatic wounds, chronic wounds, burns) included on a tissue site.

The application of negative pressure to tissue site by the therapy system can impart a pulling (e.g., lifting) force to intact skin, which decompresses the treatment tissue site and may increase the perfusion of blood and other fluids (e.g. lymphatic flow) at the treatment tissue site. This decompression may advantageously result in or accelerate the reduction of swelling at the tissue site. Additionally, application of negative pressure to a wound is known to facilitate wound healing.

According, the therapy system described herein may be configured for use in non-medical settings, and may be used to treat swelling occurring as a result of a variety of different conditions. For example, the therapy system may be used in an at-home setting by a patient to treat swelling or anticipated swelling resulting from an injury, over-exertion, an underlying medical condition (e.g., lymphedema), etc. The therapy system may also aid in recovery from underlying injuries (e.g., sprains, strains, etc.). As another example, the therapy system may be used in an athletic training, sports medicine, or athletic performance context to facilitate recovery from physical training and athletic competition (e.g., aerobic or anaerobic exertion).

In some scenarios, the therapy system is used in a medical setting, such as, e.g., to reduce swelling during pre- and/or post-operative care of a patient. For example, reducing swelling at a treatment site (e.g., caused by a broken bone, edema, tissue sprain, tissue strain, etc.) prior to surgery may advantageously facilitate access to underlying tissue at a target surgical site, reduce surgery time and/or improve the outcome of surgical treatment. Use of a therapy system according to any of the embodiments described herein prior to surgical treatment may advantageously decrease the time needed to reduce swelling at the target surgical site to an acceptable degree of swelling as compared to the time that would be required to reduce swelling using conventional methods of treating swelling. For example, use of the therapy system may reduce swelling to an acceptable degree within 3 to 7 days of initiation of treatment using the therapy system. In some scenarios, the therapy system may be used post-operatively to facilitate healing of surgical incisions, to mitigate post-operative swelling, and to potentially facilitate healing of other surgically repaired or disrupted tissue structures.

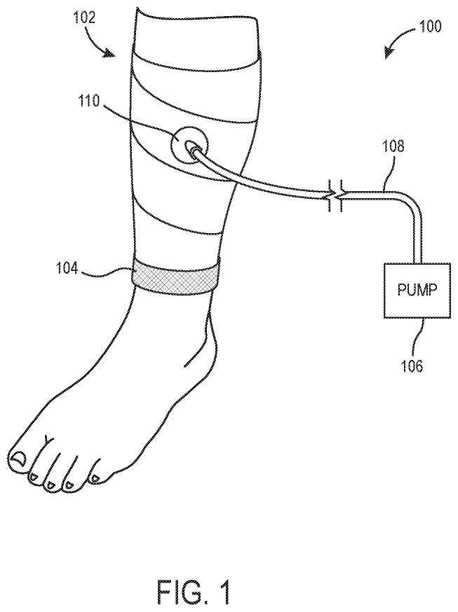

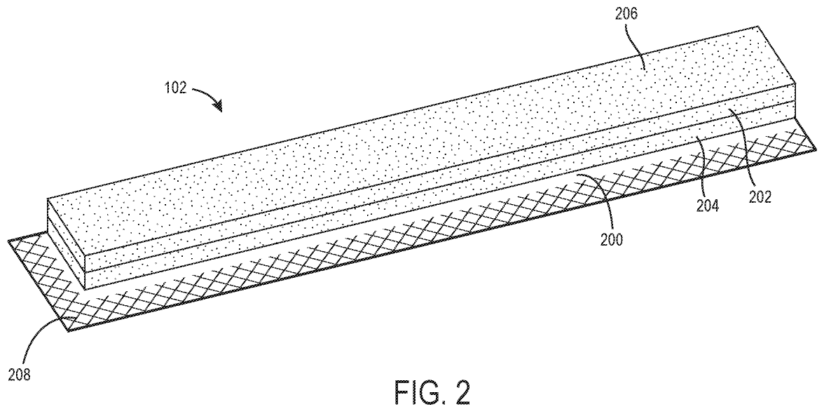

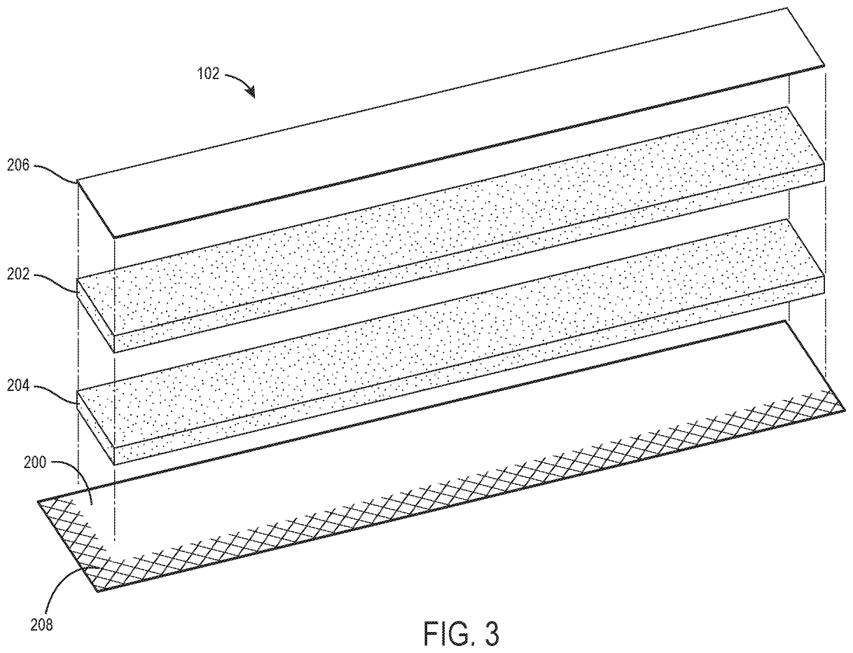

Referring to FIG. 1, a negative pressure therapy system 100 is shown, according to an exemplary embodiment. The negative pressure therapy system 100 includes a wrap (dressing, decompression wrap, etc.) 102, shown in FIG. 1 as applied to a lower leg of a patient. The wrap 102 is configured to be wrapped circumferentially around a tissue site (e.g., around a leg as shown in FIG. 1) to provide a sealed chamber defined by the wrap 102 and the tissue site. The wrap 102 is shown in detail in FIGS. 2-3 and described with reference thereto.

The wrap 102 is advantageously configured as an extend strip, for example a substantially rectangular shape having a significantly longer length than width. For example, a width of the wrap 102 may be in a range between approximately two inches and approximately five inches, for example approximately three inches, whereas a length of the wrap 102 as shown in FIG. 1 may be in a range between approximately two and four feet, for example three feet. In some embodiments, the wrap 102 may be distributed in pre-cut strips having lengths suitable for single applications to individual patients. For example, a kit may include pre-cut strips of a single length (e.g., targeting a particular anatomical region) or pre-cut strips of various lengths (e.g., allowing for application to different anatomical region or for patients of different sizes). In other embodiments the wrap 102 may be distributed having a length that provides enough material for multiple applications (e.g., for multiple patients) after cutting (or other separation) into separate wraps 102 by a caregiver. For example, the wrap 102 may be distributed as a roll having a length (when unrolled) between approximately 8 and approximately 15 feet, for example approximately 10 feet or approximately 3 meters.

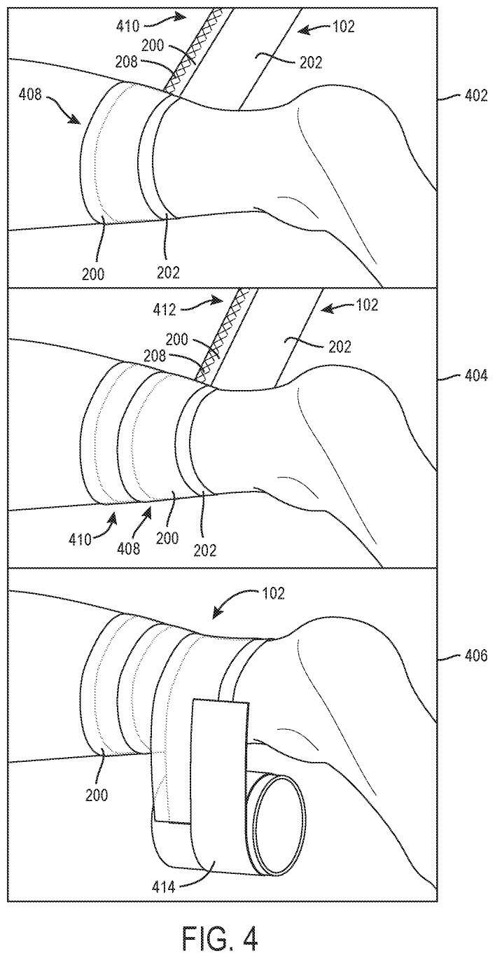

The wrap 102 is configured to be wrapped around tissue sites having various geometries, for example to provide circumferential coverage of a tissue site. An example process for applying the wrap 102 is illustrated in FIG. 4 and described with reference thereto below. FIG. 1 illustrates the wrap 102 wrapped around a lower leg of a patient. The wrap 102 could alternatively have been applied to a different anatomical region, for example an upper leg, knee joint, ankle joint, foot, groin or hip area, shoulder, upper arm, forearm, elbow, hand, neck. With a longer section of the wrap 102, torso applications are also possible. The wrap 102 is therefore advantageously configured for highly adaptive use for treatment of a wide variety of treatment sites. Additionally, application of the wrap 102 may be intuitive for caregivers who regularly apply other types of wrap-style treatments, for example compression wraps or athletic tape.

FIG. 1 shows the negative pressure therapy system 100 as including a sealing cuff 104. The sealing cuff 104 is configured to be positioned at one end of the wrap 102 when the wrap 102 is wrapped around a tissue site. The sealing cuff 104 is configured to seal the end of the wrap 102 to the patient and/or to resist migration (movement, slipping, detachment, etc.) of the wrap 102 relative to the tissue site, without compressing the tissue site or constricting internal flow of body fluids to or from the tissue site. In some embodiments, the sealing cuff 104 may be provided as a stretchy ring configured to stretch to fit over the tissue site and the wrap 102 and to rebound gently to hold in place on the tissue site without constricting or compressing the tissue site. For example, a thermoplastic elastomer or silicone ring may be provided. In some embodiments, the sealing cuff 104 is provided as a film strip with an adhesive configured to adhere to both the wrap 102 and the tissue site. In some embodiments, the sealing cuff is provides as a strap that can be positioned and tightened, for example using a hook-and-loop attachment structure or adjustable buckle. Although FIG. 1 shows the sealing cuff 104 only at a single end of the wrap 102, in some embodiments a sealing cuff 104 is also provided at a second, opposing end of the wrap 102. In other embodiments, depending on the geometry of the tissue site, a sealing strip (e.g., a film having an adhesive on one side) may be provided to seal exposed edges of the wrap 102 that are unsuitable for circumferential sealing by a closed-loop sealing cuff 104.

The negative pressure therapy system 100 also includes a negative pressure source, shown as pump 106. The pump 106 is shown as being fluidly connected to the wrap 102 via tubing 108 and a connection pad 110. In particular, the connection pad 110 provides a substantially air-tight fluid connection between the tubing 108 and a manifold layer (shown in FIGS. 2-3) of the wrap 102, i.e., such that air can flow from the manifold layer to the tubing 108 via the tubing, but ambient air is substantially prevented from leaking into the manifold layer at the connection pad 110. The tubing 108 extends from the connection pad 110 to the pump 106.

The pump 106 is operable to remove air from the wrap 102, and more particularly from a sealed chamber defined between an outermost layer of the wrap 102 and the tissue site, via the connection pad 110 and the tubing 108. By removing air from the wrap 102, the pump 106 operates to establish a negative pressure (relative to atmospheric pressure) at the wrap 102 and the tissue site. The pump 106 may operate intermittently to sustain a substantially constant negative pressure at the wrap 102, for example a negative pressure of between approximately 100 mmHg and 150 mmHg. In some embodiments, the pump 106, connection pad 110, and tubing 108 are configured such that the pump 106 can obtain measurements of a pressure at the wrap 102 and use such measurements as inputs to controlling the pump 106. In some embodiments, the pump 106 is electrically powered and includes or can be connected to an energy source (e.g., battery). In some embodiments, the pump 106 is manually actuated by a user to cause air displacement.

The negative pressure may result in decompression of the tissue site. For example, the negative pressure may result in collapse of features of the wrap 102 in a radially-outward direction away from the tissue site. The pressure differential and/or the space opened by such collapse may pull outwardly on intact skin of the tissue site, resulting in decompression of the tissue site. Such decompression may facilitate swelling reduction and/or provide other therapeutic benefits to the tissue site. In scenarios where the tissue site includes a wound, applying negative pressure at the wound may also facilitate wound healing.

FIG. 1 illustrates the pump 106, tubing 108, the connection pad 110, the sealing cuff 104, and the wrap 102 coupled together as applied to a tissue site. In some embodiments, the therapy system 100 is manufactured and distributed with the pump 106, tubing 108, and connection pad 110 already coupled to the wrap 102. In some such embodiments, the sealing cuff 104 may be provided separately (e.g., in a kit as a distinct item) or also already coupled to the wrap 102. In other embodiments, the therapy system 100 is manufactured and distributed in a kit that includes, as separate elements, the pump 106, tubing 108, connection pad 110, sealing cuff 104, and the wrap 102. In some cases, the tubing 108 may be coupled to the connection pad 110 as distributed in the kit that separately includes the pump 106 and wrap 102.

In some embodiments, the wrap 102 is distributed separately (e.g., as an extended roll including sufficient material for multiple applications) from one or more pumps 106. In other examples, a kit for may include multiple pumps and a sufficient amount of the wrap 102 for application to multiple patients. In some examples, the pump 106 may be configured for reuse whereas the wrap 102 is configured as a single-use element, and additional wraps 102 can be obtain for use with an existing pump 106. The tubing 108 and connection pad 110 may be reusable or single-use in various embodiments. In such cases, the pump 106 can be used over time for multiple therapy sessions for different patients or for different applications of the wrap 102, which may be particularly advantageous in an athletic training or at-home self-use scenario. In other examples, a kit for may include multiple pumps and a sufficient amount of the wrap 102 for application to multiple patients. In some embodiments, the wrap 102 is also configured for reuse or reapplication. In some embodiments, a single pump 106 may be suitable for simultaneous use with multiple wraps 102 applied to distinct tissue sites on a single patient.

Referring now to FIGS. 2-3, close-up views of the wrap 102 are shown, according to exemplary embodiments. FIG. 2 shows a perspective view of the wrap 102, while FIG. 3 shows an exploded view. From the perspective of FIGS. 2-3, a tissue-facing direction of the dressing is up and non-tissue-facing direction for the direction is down. That is, the wrap is configured to be applied to a tissue site such that the “top” of the dressing as illustrated on the page in FIGS. 2-3 interfaces with a tissue site and the “bottom” of the dressing as illustrated on the page in FIGS. 2-3 interfaces with an ambient environment.

As shown in FIGS. 2-3, the wrap 102 includes an occlusive layer 200 coupled to a manifold layer 202 by an adhesive layer 204, such that the adhesive layer 204 is positioned between the occlusive layer 200 and the manifold layer 202. The wrap 102 is also shown to include a patient interface layer 206 coupled to and positioned at the manifold layer 202, such that the manifold layer 202 is positioned between the patient interface layer 206 and the adhesive layer 204. When the wrap 102 is properly applied to a tissue site, the patient interface layer 206 is positioned between the manifold layer 202 and the tissue site. The occlusive layer 200, the manifold layer 202, the adhesive layer 204, and the patient interface layer 206 all have lengthwise dimensions that are substantially longer than perpendicular widthwise dimensions (e.g., greater than five time longer) to provide the wrap 102 with the dimensions described above.

The occlusive layer 200 is formed as a first strip having a longitudinal axis. The manifold layer 202 is formed as a second strip and extends parallel to the longitudinal axis of the occlusive layer 200. As shown, the adhesive layer 204 is shaped to match the overlapping area between the manifold layer 202 and the occlusive layer and defines an interface between the manifold layer and the occlusive layer 200. The manifold layer 202 is positioned relative to the occlusive layer 200 such that a first lateral side (edge) of the occlusive layer 200 is coextensive with, or coterminous with, a first lateral side of the manifold layer 202 (i.e. in a first lateral direction perpendicular to the longitudinal axis of the occlusive layer 200), and a second lateral side of the occlusive layer 200 overextends (i.e. extends beyond) the second lateral side of the manifold layer 202 (i.e. in a second lateral direction perpendicular to the longitudinal axis of the occlusive layer 200). That is, the manifold layer 202 and the occlusive layer 200 may extend in a shared lengthwise (longitudinal, longer) direction but may be offset in a widthwise (lateral, shorter) direction, with the first lateral sides of the occlusive layer 200 and the manifold layer 202 extending along a mutually common border, and the second lateral side of the occlusive layer 200 overextending the second lateral side of the manifold layer 202. For example, a portion of the occlusive layer 202 extending beyond the manifold layer 200 may have a width of less than ten centimeters, preferably in a range between two centimeters and five centimeters (i.e., as measured in the widthwise direction). In some embodiments, the manifold layer 202 extends beyond the occlusive layer 200 in a second lateral direction opposite the first lateral direction. In some embodiments, the manifold layer 202 may be narrower (i.e., have a smaller widthwise dimension) than the occlusive layer 200. As shown in FIG. 2, the occlusive layer 200 may also be longer than the manifold layer 202.

Accordingly, as shown in FIGS. 2-3, the patient-facing surface of the occlusive layer 200 includes both a portion that is covered by the adhesive layer 204 (aligned with the manifold layer 202) and an exposed portion. The wrap 102 also includes an exposed adhesive 208 provided on the exposed portion of the patient-facing surface of the manifold layer 202.

The occlusive layer 200 is substantially air-impermeable and is configured to define a substantially airtight chamber between the occlusive layer 200 and the tissue site when the wrap 102 is applied to the tissue site. The occlusive layer 200 is composed of a flexible material configured to conform to irregular geometries when the wrap 102 is wrapped around a tissue site. The occlusive layer 200 may be durable, such that the occlusive layer 200 is configured to be manipulated during application and to provide a reliable barrier through an extended wear time (e.g., several days) for the wrap 102.

The occlusive layer 200 may be provided with a small degree of flexibility to facilitate conformability while also preventing the occlusive layer 200 from exerting a compressive force on the tissue site. For example, the occlusive layer 200 may able to stretch by between approximately 5% and approximately 10%. In some embodiments, the occlusive layer 200 is configured to provide an indication that an excessive tension (e.g., greater than a threshold tension, causing greater than approximately 10% stretch) is applied to occlusive layer 200. The indication may be provided as a visual indicator (e.g., dashes/lines, color-changing section) to communicate to a user that occlusive layer 200 is being subjected to an excess tension. The indication may be provided via a tendency of the occlusive layer 200 to break (rip, tear, etc.) when subjected to an excessive tension. Such indicators may advantageously guide a user in applying the wrap 102 around a tissue site without compressing or constricting the tissue site. The occlusive layer 200 may have a high moisture vapor transfer rate (MVTR) to allow moisture (e.g. perspiration) to be evaporated from the treatment tissue site during use of the therapy system 100.

In some embodiments, the occlusive layer 200 is formed as a polyurethane film. The film may have a thickness in a range between approximately 3 millimeters and approximately 10 millimeters. In other embodiments, the occlusive layer 200 may be formed of one or more of poly alkoxyalkyl acrylates or methacrylates, laminated fabrics (e.g., polyrurethane laminated fabric, expanded polytetrafluoroethylene laminated fabric, etc.), polymer-coated fabric, or fabric made from synthetic fibers.

The adhesive layer 204 is configured to adhere the occlusive layer 200 to the manifold layer 202. The adhesive layer 204 may be formed of any suitable adhesive, for example an acrylic adhesive. In some embodiments, the adhesive layer 204 include a film (e.g., a polyurethane film) coated on both sides within an adhesive. In some embodiments, the adhesive layer 204 is composed of one or more adhesives without any intervening structures.

The manifold layer 202 is configured to provide for airflow and the communication of negative pressure throughout the manifold layer 202. The manifold layer may be configured to compress or collapse under negative pressure in a direction away from a tissue site and toward the occlusive layer 200. In such embodiments, the manifold layer 202 is configured to maintain open channels for airflow in the compress or collapsed state. Accordingly, the manifold layer 202 is formed from a material including (or defining) a plurality of flow channels (e.g. pathways, passageways, pores, etc.) therethrough. The flow channels of the manifold layer 202 allow for a sustained transmission (e.g., manifolding) of negative pressure to the treatment tissue site during operation of the therapy system 100. Some or all of the flow channels are interconnected to improve the distribution of fluids (e.g. air) removed from the treatment tissue site by a pressure differential created by the pump 106. The manifold layer 202 is formed from a compressible material having a stiffness sufficient to provide airflow through the flow channels at negative pressures up to at least approximately 150 mmHg.

The manifold layer 202 is sufficiently flexible and conformable to facilitate to conform to irregular geometries when the wrap 102 is wrapped around a tissue site. The manifold layer 202 may be provided with a degree of stretchiness to facilitate application of the wrap 102 while mitigating a risk that the wrap 102 may cause compression or constriction of the tissue site. The manifold layer 202 is configured to bend and stretch to match bending and stretching of the occlusive layer 200 to facilitate application of the wrap 102 to a tissue site.

In some embodiments, the manifold layer 202 is formed from an open-celled polyurethane foam. The polyurethane foam may have a thickness in a range between ⅛ inch and ⅜ inch, for example approximately ¼ inch. In some embodiments, the manifold layer 202 is provided with a variable density, compressibility, or other structural elements such that the manifold layer 202 is configured to collapse toward the occlusive layer 200 and away from a tissue site when subjected to negative pressure.

The patient interface layer 206 is configured to provide a comfortable interface between the wrap 102 and the tissue site. The patient interface layer 206 is configured to contact the tissue site for an extended period of time without irritation of the tissue site, ingrowth of tissue into the wrap 102, adherence between the patient interface layer 206, thereby protecting the tissue site from potential side effects of wearing and removing the wrap 102. The patient interface layer 206 is configured to wick moisture away from the tissue site and into the manifold layer 202. In some embodiments, the patient interface layer 206 is configured to provide odor reduction at the tissue site. In some embodiments, the patient interface layer 206 is also perforated to facilitate communication of negative pressure from the manifold layer 202 to the tissue site. For example, the patient interface layer 206 may be formed as a perforated silicone film. As another example, the patient interface layer 206 may be formed from a textile or porous non-woven material.

The exposed adhesive 208 is positioned on the occlusive layer 200 and is configured to seal a first longitudinal section of the occlusive layer 200 to a second longitudinal section of the occlusive layer 200 when the wrap 102 is wrapped around a tissue site, where the first longitudinal section is spaced apart from the second longitudinal section by approximately a circumference of the tissue site. That is, when the wrap 102 is wrapped around a tissue site, the exposed adhesive 208 on a patient-facing surface of the occlusive layer 200 can adhere to a non-patient facing surface of the occlusive layer 200, such that the patient-facing surface of a first longitudinal section of the occlusive layer 200 is sealed to a non-patient facing surface of a second longitudinal section of the occlusive layer 200. The occlusive layer 200 is thereby sealed to itself by the exposed adhesive 208 as the wrap 102 is applied to the dressing. The exposed adhesive 208 is configured to provide a substantially airtight seal between two sections of the occlusive layer 200, thereby facilitating creation of a substantially air-tight barrier that surrounds a tissue site. The width of the portion of the occlusive layer 200 which extends beyond the manifold layer 202 and includes the adhesive can have a width less than ten centimeters (e.g., in a range between two centimeters and five centimeters by way of example) in order to facilitate handling and to allow coupling of the occlusive layer 200 to itself without coupling of the occlusive layer 200 to the patient's skin along the longitudinal extent of the dressing. At one or both longitudinal ends of the wrap 102, the exposed adhesive 208 may also be provided at one or both ends of the wrap 102 and configured to adhere to a tissue site (e.g., to intact skin) to secure the wrap 102 in position relative to the tissue site and establish a seal between the tissue site and the occlusive layer 200.

The exposed adhesive 208 may include a flowable, gummy adhesive of a sufficient thickness to fill gaps, creases, etc. formed in the occlusive layer 200 when the wrap 102 is applied to a tissue site. The exposed adhesive is thereby configured to reduce a risk of leaks between two lateral extends of the occlusive layer 200 when the occlusive layer 200 is wrapped around a tissue site. For example, a silicone adhesive may be used. In some such embodiments, the exposed adhesive 208 is configured for repeated reuse, such that the wrap 102 can be applied (with a seal established by the exposed adhesive 208), removed by peeling the exposed adhesive 208 away from the non-patient-facing surface of a section of the occlusive layer 200, and then reapplied at a later time, to a different patient, to a different tissue site, in a different arrangement on the same tissue site, etc.

In some embodiments, a high-tackiness adhesive is used in addition to or as an alternative to the gummy adhesive. For example, in some embodiments an acrylic adhesive is included. The high-tackiness adhesive may facilitate long-time wear of the wrap 102 (e.g., up to two weeks), and may provide a substantially-permanent adhesion between the two surfaces of the occlusive layer 200. In such embodiments, removal for the wrap 102 from a tissue site may require cutting through the wrap 102 to release the wrap 102 from the patient.

Referring now to FIG. 4, a storyboard style illustration of a process 400 for applying the wrap 102 to a tissue site is shown, according to an exemplary embodiment. In particular, process 400 shows the wrap 102 being spirally wrapped (helically wrapped, wrapped in spiral configuration with the over-extending portion of occlusive layer 200 on one lateral side over-lapping the non-overextending portion of occlusive layer 200 on the opposite lateral side, spiraled) around and along the tissue site. The process 400 is illustrated in three steps, shown sequentially in a first frame 402, a second frame 404, and a third frame 406. In the example shown, the tissue site is an ankle of a patient, for example including a wound created by a surgical incisions or swollen due to traumatic injury. However, the process 400 can be adapted for application of the wrap 102 at various anatomical regions.

As shown in the first frame 402, a first longitudinal section 408 the wrap 102 is adhered to the tissue site by the exposed adhesive 208. A corresponding section of the manifold layer 202 is also positioned at the tissue site. A patient interface layer 206 may be included and obscured from view between the manifold layer 202 and the tissue site. In the example shown, the occlusive layer 200 extends beyond the manifold layer 202 in a first lateral direction (to the left from the perspective of FIG. 4), while the manifold layer 202 extends beyond the occlusive layer 200 in a second lateral direction (to the right from the perspective of FIG. 4). Accordingly, a portion of the occlusive layer 200 is not covered by the manifold layer 202, while a portion of the manifold layer 202 is not covered by the occlusive layer 200.

The first frame 402 further shows a second longitudinal section 410 of the wrap 102. As shown, the wrap 102 has been wrapped (curved, bent, curled, etc.) around the tissue site, such that the second longitudinal section 410 of the wrap 102 is set to overlap the first longitudinal section 408 of the wrap 102. As shown, the wrap 102 is applied in a helix or spiral shape. That is, the wrap 102 extends at an angle around the tissue site such that the second longitudinal section 410 of the wrap 102 is laterally offset from the first longitudinal section 408 of the wrap 102. That is, as shown in frame 402 of FIG. 4, the second longitudinal section 410 of the wrap 102 is positioned to partially overlap the first longitudinal section 408 of the wrap 102 while also being partially aligned with a portion of the tissue site not yet covered by the wrap 102.

To transition from the first frame 402 to the second frame 404, the occlusive layer 200 of the second longitudinal section 410 of the wrap 102 is sealed to the occlusive layer 200 of the first longitudinal section 408 of the wrap 102 using the exposed adhesive 208 on the patient-facing surface of the occlusive layer 200 of the second longitudinal section 410 of the wrap 102. In other words, the exposed adhesive 208 on the underside of the upper of the two sections of the occlusive layer 200 is used to bind the occlusive layer 200 to itself. This results in creation of a substantially continuous occlusive layer 200 extending along and around the tissue site.

As illustrated in FIG. 4, the transition step from the first frame 402 to the second frame 404 also includes placing the manifold layer 202 of the second longitudinal section 410 of the wrap 102 in contact with the manifold layer 202 of the first longitudinal section 408 of the wrap 102. This results in a substantially continuous manifold layer 202 extending along and around the tissue site.

The second frame 404 shows the first longitudinal section 408 of the wrap 102 sealed over the second longitudinal section 410 of the wrap 102 and wrapped around the tissue site. The second longitudinal section 410 partially overlaps the first longitudinal section 408, with the remainder in contact with the tissue site. The second frame 404 illustrates that the wrap 102 can continue to be wrapped around the tissue site. A third longitudinal section 412 of the wrap 102 is shown, similarly wrapped around and laterally offset as described above with reference to the first frame 402. The process 400 includes sealing the occlusive layer 200 of the third longitudinal section 412 of the wrap 102 to the occlusive layer 200 of the second longitudinal section 410 with the exposed adhesive 208 of the third longitudinal section 412. The process 400 also includes placing the manifold layer 202 of the third longitudinal section 412 in contact with the manifold layer 202 of the second longitudinal section 410. A substantially-continuous occlusive layer 200 is thereby formed around a substantially-continuous manifold layer 202 that extends along and around the tissue site.

The third frame 406 shows continued application of the wrap 102 to the tissue site. The wrap 102 can be applied in this manner until a desired amount of the tissue site is covered with the wrap 102 or a predetermine length of the wrap 102 has been applied to the tissue site. The third frame 406 also shows that the wrap 102 may be distributed with a backing strip 414 that prevents premature coupling of the exposed adhesive 208 to other structures, and can be removed and discarded during application of the wrap 102 to expose the exposed adhesive 208. Preferably, the wrap 102 is applied with a minimal amount of tension in the wrap 102, and such that the wrap 102 does not apply a compressive force on the tissue site.

Once the wrap 102 has been fully applied the tissue site, an open end of the wrap 102 (the end to the right in FIG. 4) may not self-adhere directly to the tissue site due to positioning of the manifold layer 202. The sealing cuff 102 can be applied around this open end to establish a seal between the occlusive layer 200 and the tissue site around the open end. A substantially airtight chamber can thereby be established between the occlusive layer 200 and the tissue site which houses the manifold layer 202. Because the wrap 102 has been spiral wrapped around and along the tissue site, the chamber has a width (i.e., a dimension along the tissue site in a width-wise direction of the wrap 102, up or down the leg in the example shown) which is greater than a width of the occlusive layer 200 at any given point on the wrap 102.

In some embodiments, the connection pad 110, tubing 108, and pump 106 are pre-coupled to the wrap 102. In other embodiments, applying the dressing includes coupling the connection pad 110 to the wrap 102 in a desired position, for example selected to minimize interference with a patient's ordinary movement and to facilitate application of negative pressure throughout the manifold layer. To apply the connection pad 110, a hole is created in the occlusive layer at the desired position for the connection pad 110. The connection pad 110 is then coupled to the occlusive layer 200 to cover the hole, for example using adhesive provided on the connection pad 110. The connection pad 110 is thereby placed in fluid communication with the manifold layer 202. The tubing 108 can be connected to the connection pad 110 and the pump can be connected to the pump 106 to place the pump 106 in fluid communication with the manifold layer 202 to complete set-up of the therapy system 100. The pump 106 can then be operated to remove air form the manifold layer 202, creating a negative pressure at the tissue site that may provide the therapeutic benefits described above.

### Configuration of Exemplary Embodiments

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

1. A decompression wrap, comprising:
an occlusive layer formed as a first strip having a longitudinal axis;
a manifold layer coupled to the occlusive layer and formed as a second strip extending parallel to the longitudinal axis, wherein the occlusive layer extends beyond the manifold layer in a first lateral direction perpendicular to the longitudinal axis without extending beyond the manifold layer in a second lateral direction opposite the first lateral direction; and
an adhesive provided on the occlusive layer and configured to seal a first longitudinal section of the occlusive layer to a second longitudinal section of the occlusive layer when the decompression wrap is spiral wrapped around a tissue site.

2. The decompression wrap of claim 1, wherein the occlusive layer extends beyond the manifold layer in the first lateral direction by less than ten centimeters.

3. The decompression wrap of claim 1, wherein the occlusive layer extends beyond the manifold layer in the first lateral direction by a distance in a range between two centimeters and five centimeters.

4. (canceled)

5. The decompression wrap of claim 1, wherein the manifold layer comprises an open-celled foam and the occlusive layer is substantially air-impermeable.

6. The decompression wrap of claim 1, wherein the manifold layer extends beyond the occlusive layer in the second lateral direction.

7. The decompression wrap of claim 1, wherein:
the manifold layer comprises a first portion longitudinally offset from a second portion of the manifold layer; and
the first portion contacts the second portion when the first longitudinal section of the occlusive layer is sealed to the second longitudinal section of the occlusive layer by the adhesive.

8. The decompression wrap of claim 1, further comprising a patient interface layer coupled to the manifold layer such that the manifold layer is positioned between the patient interface layer and the occlusive layer.

9. (canceled)

10. The decompression wrap of claim 1, wherein the manifold layer is configured to collapse toward the occlusive layer when a negative pressure is established at the manifold layer.

11. A negative pressure therapy system, comprising:
a wrap, comprising:
an occlusive layer formed as a first strip having a longitudinal axis;
a manifold layer coupled to the occlusive layer and formed as a second strip extending parallel to the longitudinal axis, wherein a portion of the occlusive layer extends beyond the manifold layer in a first lateral direction perpendicular to the longitudinal axis, wherein the portion of the occlusive layer extending beyond the manifold layer has a width less than ten centimeters; and
an adhesive provided along the portion of the occlusive layer extending beyond the manifold layer; and
a pump configured to be placed in fluid communication with the manifold layer and operable to remove air from the manifold layer.

12. (canceled)

13. The negative pressure therapy system of claim 11, wherein the occlusive layer does not extend beyond the manifold layer in a second lateral direction opposite the first lateral direction.

14. The negative pressure therapy system of claim 11, wherein the wrap is configured to be spiral wrapped around a tissue site such that a first longitudinal section of the occlusive layer is sealed to a second longitudinal section of the occlusive layer by the adhesive and the manifold layer is positioned between the occlusive layer and the tissue site.

15. The negative pressure therapy system of claim 14, wherein the manifold layer comprises a first portion longitudinally offset from a second portion of the manifold layer; and wherein the first portion contacts the second portion when the first longitudinal section of the occlusive layer is sealed to the second longitudinal section of the occlusive layer by the adhesive.

16. The negative pressure therapy system of claim 11, further comprising a sealing ring configured to provide a substantially air-tight seal between a first end of the wrap and a tissue site.

17. The negative pressure therapy system of claim 11, wherein the wrap is configured to be applied to a tissue site such that a chamber is defined between the occlusive layer and the tissue site, wherein the manifold layer is positioned in the chamber and the pump is in fluid communication with the chamber and configured to establish a negative pressure in the chamber.

18.-19. (canceled)

20. The negative pressure therapy system of claim 17, wherein the manifold layer is configured to collapse away from the tissue site when the negative pressure is established in the chamber.

21. The negative pressure therapy system of claim 11, wherein the manifold layer comprises an open-celled foam and the occlusive layer is substantially air-impermeable.

22. (canceled)

23. A method for applying decompression therapy, comprising:
obtaining a wrap comprising an occlusive layer and a manifold layer;
spirally wrapping the wrap around and along a tissue site such that the occlusive layer defines a chamber between the occlusive layer and the tissue site, the manifold layer positioned in the chamber and the chamber having a width greater than a width of the occlusive layer;
removing air from the chamber with a pump to establish a negative pressure at the manifold layer.

24.-26. (canceled)

27. A kit, comprising:
a plurality of decompression wraps, each decompression wrap comprising:
an occlusive layer;
a manifold layer coupled to the occlusive layer and coextensive with the occlusive layer along a first lateral side parallel to a longitudinal axis of the occlusive layer and wherein at least a portion of the occlusive layer extends beyond the manifold layer at a second lateral side opposite the first lateral side; and
an adhesive provided along the portion of the occlusive layer extending beyond the manifold layer, wherein the adhesive is configured to seal a first portion of the occlusive layer to a second portion of the occlusive layer when the decompression wrap is applied to a tissue site; and
a pump configured to be placed in fluid communication with the manifold layer and operable to remove air from the manifold layer.

28. The kit of claim 27, wherein the plurality of decompression wraps are provided as a continuous roll of material configured to be selectively divided by a user into the plurality of decompression wraps.

29. (canceled)

30. The kit of claim 27, wherein the plurality of decompression wraps have a plurality of different predetermined lengths.

31. The kit of claim 27, further comprising tubing configured to be coupled to the pump and the wrap by a user.

32. The kit of claim 31, wherein the tubing is coupled to a connection pad configured to facilitate coupling of the tubing to the wrap by the user.

33.-34. (canceled)

35. The kit of claim 27, further comprising an adhesive sealing strip configured to facilitate selectively establishing an airtight chamber between one of the plurality of wraps and a patient site.

36. (canceled)
